(19) United States
(12) Patent Application Publication
(10) Pub. No.: US 2017/0091411 A1
(43) Pub. Date: Mar. 30, 2017

(54) SYSTEMS AND METHODS FOR SERIAL TREATMENT OF A MUSCULAR-SKELETAL DEFORMITY

(71) Applicant: Vanderbilt University, Nashville, TN (US)

(72) Inventors: Jonathan Schoenecker, Nashville, TN (US); Nathaniel Braman, Bethel Park, PA (US); Attiyya Houston, Lilburn, GA (US); Melena Mendive, San Antonio, TX (US); Simeng Miao, Columbus, OH (US)

(21) Appl. No.: 15/275,690

(22) Filed: Sep. 26, 2016

Related U.S. Application Data
(60) Provisional application No. 62/222,972, filed on Sep. 24, 2015.

(51) Int. Cl. G06F 19/00; A61F 5/01; B29C 67/00; B33Y 50/02; B33Y 10/00; B33Y 30/00; A61B 5/00; G05B 17/02

(52) U.S. Cl. CPC G06F 19/3437; A61B 5/4538; A61F 5/0111; G06F 19/3456; G05B 17/02; G06F 19/321; B33Y 50/02; B33Y 10/00; B33Y 30/00; B29C 67/0088

(57) ABSTRACT

Systems and methods for establishing a serial treatment plan to correct a muscular-skeletal deformity of a subject are described herein. A method can include receiving an image of a portion of the subject's body having the muscular-skeletal deformity, processing the image of the portion of the subject's body to establish a quantitative measure of the muscular-skeletal deformity, and determining a therapeutic state to correct the muscular-skeletal deformity. The therapeutic state can include an adjustment of the portion of the subject's body. In addition, at least one characteristic of the adjustment of the portion of the subject's body can be related to the quantitative measure of the muscular-skeletal deformity.

### CROSS-REFERENCE TO RELATED APPLICATIONS

This application claims the benefit of U.S. Provisional Patent Application No. 62/222,972, filed on Sep. 24, 2015, entitled “SYSTEMS AND METHODS FOR SERIAL TREATMENT OF A MUSCULAR-SKELETAL DEFORMITY,” the disclosure of which is expressly incorporated herein by reference in its entirety.

### BACKGROUND

Clubfoot is a congenital deformity that affects approximately 1 in 1,000 live births and is characterized by four components—cavus, adduction, varus, and/or equinus (CAVE). The most common conventional treatment option is the Ponseti method, which incorporates serial castings (e.g., weekly castings) that stretch and manipulate the foot into the correct position. However, conventional clubfoot treatment can be inconvenient and inefficient. For example, the number of physicians specializing in clubfoot treatment is relatively small, forcing many patients to travel long distances on a regular basis. Typically, the patient's foot is repositioned and recast during a weekly visit to the physician. Studies have shown that more frequent casting (e.g., up to 3 times per week) allows for a shorter overall span of treatment but more frequent physician visits may not be a practical option for the patient.

### SUMMARY

An example system for establishing a serial treatment plan to correct a muscular-skeletal deformity of a subject is described herein. The system can include a computing device having a processor and a memory operably coupled to the processor. It should be understood that the computing device can be configured to perform one or more of the steps of the example methods described herein. For example, the computing device can be configured to receive an image of a portion of the subject's body having the muscular-skeletal deformity, process the image of the portion of the subject's body to establish a quantitative measure of the muscular-skeletal deformity, and determine a therapeutic state to correct the muscular-skeletal deformity. The therapeutic state can include an adjustment of the portion of the subject's body. In addition, at least one characteristic of the adjustment of the portion of the subject's body can be related to the quantitative measure of the muscular-skeletal deformity. Optionally, the muscular-skeletal deformity is clubfoot.

Optionally, the image of the portion of the subject's body can be received from a remote computing device over a network. Additionally and optionally, the system can include a remote computing device communicatively connected to the computing device over a network. The remote computing device can be configured to capture the image of the portion of the subject's body. Optionally, in some configurations, the remote computing device can be a smartphone or tablet computer, for example.

Optionally, the system can include a 3D printing device communicatively connected to the computing device over a network. The 3D printing device can be configured to print a therapeutic device for application to the portion of the subject's body.

Optionally, the system can include a scoring sleeve with a marker arranged to aid in establishing the quantitative measure of the muscular-skeletal deformity. The scoring sleeve can be designed to be worn on the portion of the subject's body.

An example method for establishing a serial treatment plan to correct a muscular-skeletal deformity of a subject is described herein. The method can include receiving an image of a portion of the subject's body having the muscular-skeletal deformity, processing the image of the portion of the subject's body to establish a quantitative measure of the muscular-skeletal deformity, and determining a therapeutic state to correct the muscular-skeletal deformity. The therapeutic state can include an adjustment of the portion of the subject's body. In addition, at least one characteristic of the adjustment of the portion of the subject's body can be related to the quantitative measure of the muscular-skeletal deformity. Optionally, the muscular-skeletal deformity is clubfoot.

For example, a characteristic of the adjustment of the portion of the subject's body can be an amount, a rate, or a type of adjustment of the portion of the subject's body. Optionally, the type of adjustment can be an adjustment for correcting a particular component of the muscular-skeletal deformity (e.g., the cavus, adduction, varus, and/or equinus components of a clubfoot deformity). Alternatively or additionally, the type of adjustment can optionally be the order of adjustments for correcting the muscular-skeletal deformity (e.g., the order of correcting the cavus, adduction, varus, and/or equinus components of a clubfoot deformity).

Optionally, the method can include determining a plurality of therapeutic states to correct the muscular-skeletal deformity. Each therapeutic state can include a respective incremental adjustment of the portion of the subject's body.

Optionally, the method can include applying a therapeutic device to the portion of the subject's body to achieve the therapeutic state. For example, a therapeutic device can be a brace or cast.

Optionally, the method can include generating a 3D model image of the portion of the subject's body from the image of the portion of the subject's body, manipulating the 3D model image of the portion of the subject's body in accordance with the therapeutic state, and designing a therapeutic device for application to the portion of the subject's body to achieve the therapeutic state based on the manipulated 3D model image. Additionally and optionally, the method can include receiving a second image of the portion of the subject's body having the muscular-skeletal deformity, processing the second image of the portion of the subject's body to establish an updated quantitative measure of the muscular-skeletal deformity, determining an adjusted therapeutic state to correct the muscular-skeletal deformity, manipulating the 3D model image of the portion of the subject's body in accordance with the adjusted therapeutic state, and designing an adjusted therapeutic device for application to the portion of the subject's body to achieve the adjusted therapeutic state based on the manipulated 3D model image.

Optionally, the method can include manipulating the 3D model image of the portion of the subject's body in accordance with a parameter unrelated to the muscular-skeletal deformity, and designing an adjusted therapeutic device for application to the portion of the subject's body based on the manipulated 3D model image. For example, the parameter can account for growth of the subject and/or to otherwise adjust the therapeutic device for comfort of the subject.

Optionally, the 3D model image of the portion of the subject's body can be manipulated along an axis that is specific to the muscular-skeletal deformity. Alternatively or additionally, the 3D model image of the portion of the subject's body can optionally be simultaneously manipulated along a plurality of axes that are specific to the muscular-skeletal deformity.

Optionally, the method can include generating a surface pattern of the manipulated 3D model image of the portion of the subject's body. The therapeutic device for application to the portion of the subject's body can be designed based on the surface pattern.

Optionally, the method can include generating a 3D printing design model based on the surface pattern, and transmitting the 3D printing design model to a 3D printing device. Additionally and optionally, the method can include printing the therapeutic device based on the 3D printing design model using the 3D printing device.

Optionally, the therapeutic device can be customized for the portion of the subject's body.

Optionally, the quantitative measure can include an angular measure of the muscular-skeletal deformity.

Optionally, the image of the portion of the subject's body can include a scoring sleeve that covers the portion of the subject's body. The scoring sleeve can include a marker arranged to aid in establishing the quantitative measure of the muscular-skeletal deformity. Additionally and optionally, the method can include providing the scoring sleeve to the subject. Additionally and optionally, the step of processing the image of the portion of the subject's body to establish a quantitative measure of the muscular-skeletal deformity can include tracing a line that approximates the marker of the scoring sleeve within the image of the portion of the subject's body, and calculating an angular measure of the deformity based on the traced line. Optionally, the traced line can define at least two ends, and the angular measure is an angle between the at least two ends of the traced line.

Optionally, the muscular-skeletal deformity can include a plurality of components of deformity (e.g., the cavus, adduction, varus, and/or equinus components of a clubfoot deformity). Additionally and optionally, a respective quantitative measure can be assigned for each of the plurality of components of deformity. Additionally and optionally, the method can include receiving a plurality of images of the portion of the subject's body having the muscular-skeletal deformity. Each of the images can capture a respective one of the plurality of components of deformity. Additionally and optionally, the method can include transmitting instructions for capturing the plurality of images of the portion of the subject's body to a remote computing device. Optionally, the instructions for capturing the plurality of images of the portion of the subject's body can be visually displayed on the remote computing device.

Another system for establishing a serial treatment plan to correct a muscular-skeletal deformity of a subject is described herein. The system can include a computing device having a processor and a memory operably coupled to the processor. It should be understood that the computing device can be configured to perform one or more of the steps of the example methods described herein. For example, the computing device can be configured to receive data for constructing a three-dimensional (3D) model of a portion of the subject's body having the muscular-skeletal deformity, generate a 3D model of the portion of the subject's body from the data, establish a quantitative measure of the muscular-skeletal deformity based on the 3D model of the portion of the subject's body, and determine a therapeutic state to correct the muscular-skeletal deformity. The therapeutic state can include an adjustment of the portion of the subject's body. In addition, at least one characteristic of the adjustment of the portion of the subject's body can be related to the quantitative measure of the muscular-skeletal deformity. Optionally, the muscular-skeletal deformity is clubfoot.

Optionally, the data for constructing the 3D model of the portion of the subject's body is received from a 3D scanner. Additionally and optionally, the system can include a 3D scanner communicatively connected to the computing device over a network. The 3D scanner can be configured to obtain the data for constructing the 3D model of the portion of the subject's body.

Optionally, the system can include a 3D printing device communicatively connected to the computing device over a network. The 3D printing device can be configured to print a therapeutic device for application to the portion of the subject's body.

Optionally, the system can include a scoring sleeve with a marker arranged to aid in establishing the quantitative measure of the muscular-skeletal deformity. The scoring sleeve can be designed to be worn on the portion of the subject's body.

Another example method for establishing a serial treatment plan to correct a muscular-skeletal deformity of a subject is described herein. The method can include receiving data for constructing a three-dimensional (3D) model of a portion of the subject's body having the muscular-skeletal deformity, generating a 3D model of the portion of the subject's body from the data, establishing a quantitative measure of the muscular-skeletal deformity based on the 3D model of the portion of the subject's body, and determining a therapeutic state to correct the muscular-skeletal deformity. The therapeutic state can include an adjustment of the portion of the subject's body. In addition, at least one characteristic of the adjustment of the portion of the subject's body can be related to the quantitative measure of the muscular-skeletal deformity. Optionally, the muscular-skeletal deformity is clubfoot. Optionally, the data can be obtained by a 3D scanner.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an example system for establishing a serial treatment plan to correct a muscular-skeletal deformity of a subject according to implementations described herein.

FIG. 2 is a flow diagram illustrating example operations for establishing a serial treatment plan to correct a muscular-skeletal deformity of a subject according to implementations described herein.

FIG. 3 illustrates an example where four images are captured with a smartphone, one image for each component of the clubfoot deformity.

FIG. 4A illustrates an example scoring sleeve according to implementations described herein.

FIG. 4B is a diagram illustrating an example of locations where markers can be arranged on the scoring sleeve used to treat clubfoot.

FIG. 4C is another diagram illustrating an example of locations where markers can be arranged on the scoring sleeve used to treat clubfoot.

FIG. 5 is an example computing device.

FIG. 6A is a flow diagram illustrating example operations for calculating a quantitative measure of a clubfoot deformity using images according to implementations described herein.

FIG. 6B shows example images of a phantom foot having a clubfoot deformity and corresponding histograms.

FIG. 7A is screen captures illustrating manipulation of a 3D model of the subject's foot around an axis specific to the clubfoot deformity.

FIG. 7B is screen captures illustrating manipulation of a 3D model of the subject's foot around the subject's joints.

FIG. 8 illustrates an example therapeutic device designed based on a 3D model of the subject's foot according to implementations described herein.

FIG. 9A illustrates a Pirani scoring guide showing images of the least and most severe scoring CLB deformities (a “0” and “1” on the Pirani scale, respectively).

FIG. 9B illustrates the Pirani scoring guide with increased contrast between foot and background and removal of a finger in one of the images.

FIG. 9C illustrates the Pirani scoring guide of FIG. 9A after circle detection and segmentation of the foot.

FIG. 9D is graphs illustrating estimated CLB of the images in the Pirani scoring guide of FIG. 9A.

FIG. 10 illustrates images of a phantom foot having a clubfoot deformity with a scoring sleeve and corresponding extraction of markers from the images, which are used to calculate angles between axes of interest (e.g., a quantitative measure of clubfoot deformity).

FIG. 11 illustrates a 3D reconstruction (e.g., a 3D model) of a phantom foot having clubfoot deformity.

FIG. 12 illustrates a phantom foot having a clubfoot deformity (left image) and a 3D model of the phantom foot (right image) scaled spatially.

FIGS. 13A-13D illustrate a scoring sleeve imaged for each component of the deformity. FIG. 13A illustrates the cavus deformity. FIG. 13B illustrates the adductus deformity. FIG. 13C illustrates the varus deformity. FIG. 13D illustrates the equinus deformity.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. While implementations will be described for establishing a serial treatment plan to correct clubfoot, it will become evident to those skilled in the art that the implementations are not limited thereto, but are applicable for establishing a serial treatment plan to correct other muscular-skeletal deformities including, but not limited to, casting minor broken bones, casting prosthetic sockets, etc. and/or to provide telemedicine in remote and/or underserved areas (e.g., developing countries).

Systems and methods are described herein for clubfoot treatment through the generation of custom 3D-printed corrective clubfoot casts. The systems and methods analyze images of a subject's deformed foot with a scoring sleeve, and relay information about the extent of deformity to a predictive treatment model. The predictive treatment model then recommends corrective measures for the deformity by predicting the next stage of treatment. This treatment can be applied to a 3D model of the subject's foot, and a cast can be generated from this corrected model. As a result, patients can be provided with more than just a cast as they would be receiving a personalized therapy.

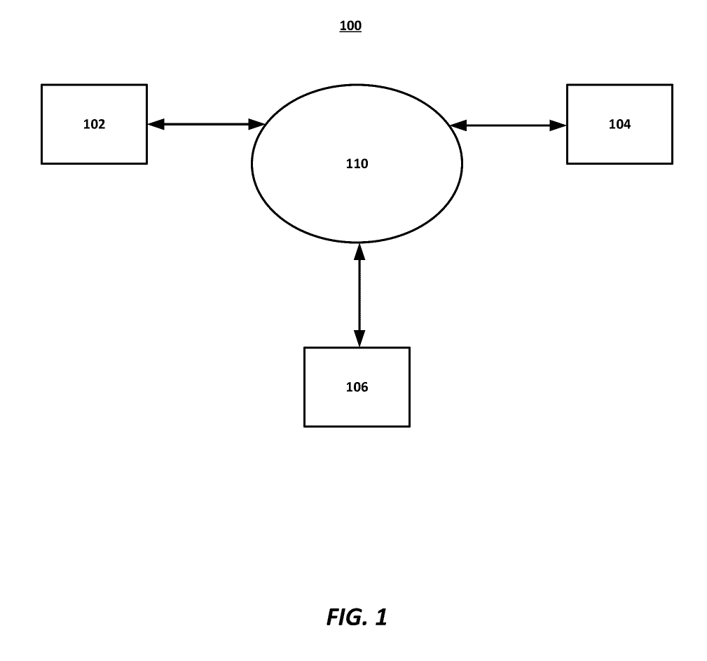

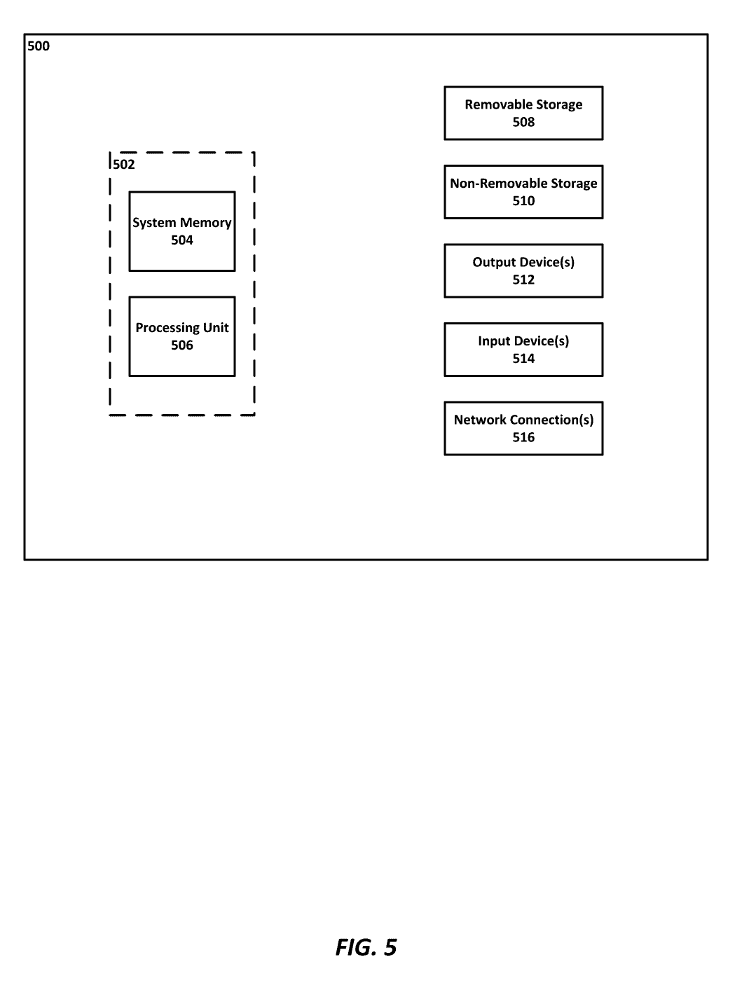

Referring now to FIG. 1, a system 100 for establishing a serial treatment plan to correct a muscular-skeletal deformity of a subject is described. The system 100 can include a computing device 102. Optionally, the computing device 102 can be a desktop computer, a laptop computer, a tablet computer, a server computer, a smartphone, or any other computing device. In some implementations, the system 100 can include a remote computing device 104. Optionally, the remote computing device 104 can include an image capturing device (e.g., a camera). As described below, the image capturing device can be configured to capture one or more images of a subject. Image capturing devices such as a camera are well known in the art and therefore not described in further detail below. For example, the remote computing device 104 can optionally be a smartphone or a tablet computer with an image capturing device. This disclosure contemplates that the images can be still images or videos (e.g., moving images). For example, the image capturing device can be a smartphone camera, which can be configured to capture both still images and videos. In other implementations, the remote computing device 104 can be a 3D scanner. A 3D scanner is device that analyzes an object (e.g., using light, sound, radiation, or other means) to collect data concerning the object's shape and sometimes appearance (e.g., color). This data can be used to construct a 3D model of the object. 3D scanners are well known in the art and therefore not described in further detail below. It should be understood that the computing device 102 and/or the remote computing device 104 can include one or more of the components of the example computing device described below with regard to FIG. 5.

Additionally and optionally, the system 100 can include a 3D printer 106. An example 3D printer is MAKERBOT 2.0 from MAKERBOT INDUSTRIES, LLC of BROOKLYN, N.Y. It should be understood that MAKERBOT 2.0 is provided only as an example and that other 3D printers can be used. A 3D printer 106 is a device configured to produce a 3D object through an additive process in which successive layers of material are deposited. 3D printers can produce objects having almost any shape and/or geometry. As described below, the 3D printer 106 can be configured to produce a therapeutic device (e.g., a brace or cast) based on a 3D model of a portion of the subject's body. 3D printers are well known in the art and are therefore not described in further detail below.

As shown in FIG. 1, the computing device 102, the remote computing device 104, and the 3D printer 106 are communicatively connected via a network 110. This disclosure contemplates that the network 110 is any suitable communication network. The network 110 can include a local area network (LAN), a wireless local area network (WLAN), a wide area network (WAN), a metropolitan area network (MAN), a virtual private network (VPN), etc., including portions or combinations of any of the above networks. The network elements and devices described herein can be coupled to the network 110 through one or more communication links. This disclosure contemplates the communication links are any suitable communication link. For example, a communication link may be implemented by any medium that facilitates data exchange between the network elements including, but not limited to, wired, wireless and optical links. Example communication links include, but are not limited to, a LAN, a WAN, a MAN, Ethernet, the Internet, or any other wired or wireless link such as WiFi, WiMax, 3G or 4G.

One or more of the computing device 102, the remote computing device 104, and the 3D printer 106 can be located in the same physical location (e.g., geographic location). For example, in some implementations, the computing device 102 and the 3D printer 106 can be located in the same physical location such as a medical facility, and the remote computing device 104 can be located at a different physical location such as the subject's home. Optionally, each of the computing device 102, the remote computing device 104, and the 3D printer 106 can be located in different physical locations. For example, in some implementations, the computing device 102 can be located in a medical facility, the remote computing device 104 can be located at a different physical location such as the subject's home, and the 3D printer 106 can be located at yet another physical location such as a printing facility. It should be understood that the system 100 described with respect to FIG. 1 is provided only as an example.

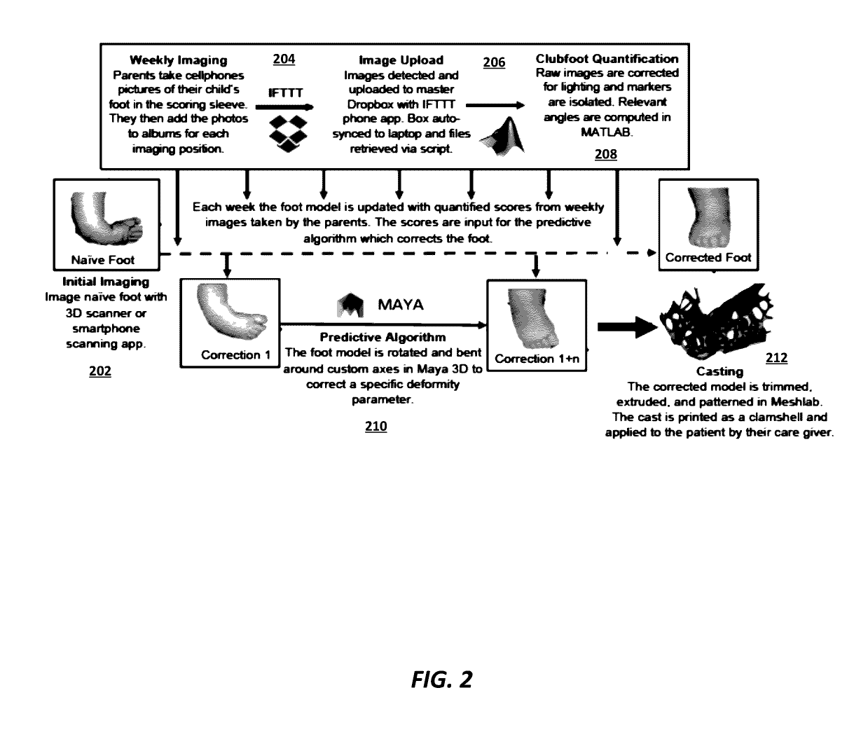

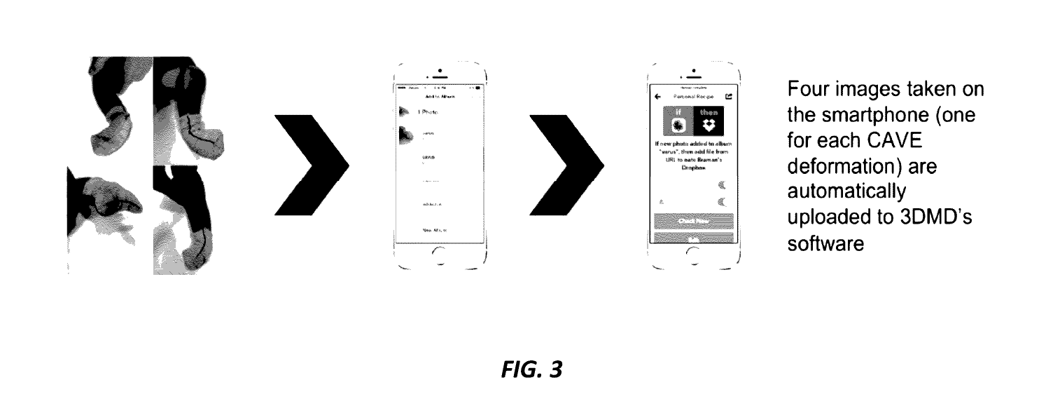

Referring now to FIG. 2, a flow diagram illustrating example operations for establishing a serial treatment plan to correct a muscular-skeletal deformity of a subject is described. In FIG. 2, the muscular-skeletal deformity is clubfoot. It should be understood, however, that the operations can be used to correct other muscular-skeletal deformities. At 202, at least one image of a portion of a subject's body having a muscular-skeletal deformity (e.g., the subject's foot) is captured, for example, using an image capturing device (e.g., the remote computing device 104 of FIG. 1). It should be understood that the at least one image can be one or more still images and/or a video. As described below, the subject can optionally be wearing a scoring sleeve on the subject's foot. The scoring sleeve can include one or more markers arranged to aid in establishing the quantitative measure of the muscular-skeletal deformity. In some implementations, a plurality of images of the subject's foot are captured. For example, FIG. 3 illustrates an example where four images are captured with a smartphone, one image for each component of the clubfoot deformity. In FIG. 3, using an application running on the smartphone, the images are uploaded from the smartphone and transferred to a computing device configured to establish the serial treatment plan. Optionally, the application running on the smartphone and/or the computing device configured to establish the serial treatment plan can provide instructions for guiding the user through the imaging process. Alternatively or additionally, in some implementations, data for constructing a 3D model of the subject's foot are obtained, for example, using a 3D scanner (e.g., the remote computing device 104 of FIG. 1).

Referring again to FIG. 2, at 204, the image is transferred to a computing device configured to establish the serial treatment plan (e.g., the computing device 102 of FIG. 1). It should be understood that the image can be one or more still images and/or a video. When the image is a video, this disclosure contemplates that a single frame can be pulled or selected from within the video, for example, based on how easily a quantitative measure can be established. For instance, it is possible to analyze the video and select a frame with adequate characteristics (e.g., using a computing device) such as lighting, lack of movement, angle, etc. In other words, frames with poor lighting, excessive movement, and/or incorrect angles can be eliminated and not used for establishing the serial treatment plan. Alternatively or additionally, multiple frames of a video can be compared, for example, in the case of deformity components where a three-dimensional approach is needed. It should be understood that using a video has advantages when dealing with the challenge of having a baby stay motionless during the image capture process. In some implementations, the image is transferred over a network (e.g., the network 110 of FIG. 1). For example, the image can optionally be acquired using a smartphone or tablet computer. Additionally, an application running on the smartphone or tablet computer can provide instructions to the user (i.e., guide) the user through the process of capturing the image (e.g., one or more still images or a video). When capturing a video, the application running on the smartphone can instruct the user to follow a “path” (e.g., start filming on the left side of the foot and rotate the camera around the heel). Additionally, an application running on the smartphone or tablet can optionally store the image at a predetermined location, for example, a storage location accessible over a network. Optionally, the predetermined location can be a cloud storage folder. DROPBOX, INC. of SAN FRANCISCO, Calif. is an example cloud storage provider. Cloud storage is well known in the art and therefore is not described further herein. Alternatively, in some implementations, the image is transferred directly to the computing device, for example, using tangible storage media.

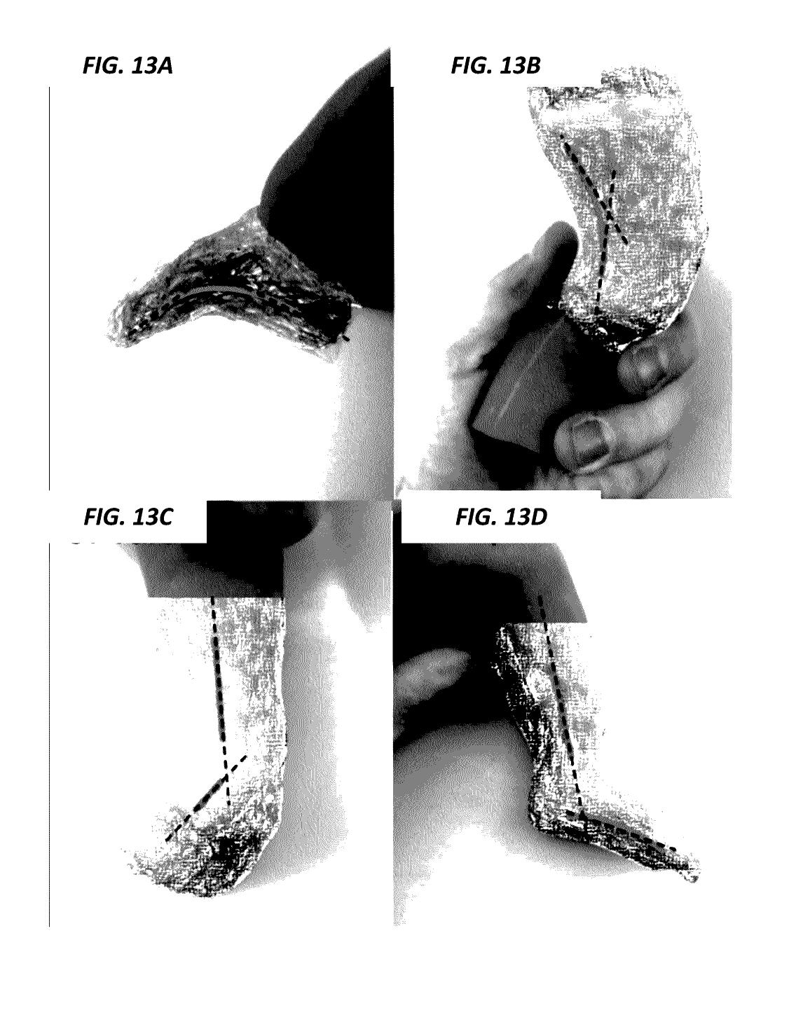

At 206, the image is received by the computing device configured to establish the serial treatment plan. Optionally, in some implementations, the computing device is configured to synchronize with the predetermined location where the image is stored by the smartphone or tablet computer (e.g., a cloud storage folder). In this implementation, the computing device can optionally retrieve the image automatically, for example, via a script or application program running on the computing device. At 208, the image is processed by the computing device to establish a quantitative measure of the muscular-skeletal deformity (e.g., clubfoot). The processing can optionally include the 3DMD Parametric Clubfoot Analysis Process (PCAP) described below. Optionally, as described below, the quantitative measure of clubfoot can be an angular measure. For example, clubfoot is characterized by a plurality of components of the deformity (e.g., the cavus, adduction, varus, and/or equinus components as shown in FIGS. 13A-13D), and the angular measure can quantitatively measure the degree or extent of one or more of the components of the deformity. This disclosure contemplates that the quantitative measure can be something other than an angular measure, which is provided only as an example. In other words, this disclosure contemplates that the quantitative measure can be any measure that quantifies the degree, extent, or severity of the deformity. For example, in some implementations below, the quantitative measure can be a measure of curvature.

At 210, a therapeutic state to correct the muscular-skeletal deformity (e.g., clubfoot) is determined by the computing device. The processing can optionally include the 3DMD Clubfoot Correction Predictive Process (CCPP) described below. The therapeutic state can include an adjustment of the portion of the subject's body (e.g., the subject's foot). Additionally, at least one characteristic of the adjustment (e.g., the amount, rate, or type of adjustment) of the portion of the subject's body can be related to the quantitative measure obtained in step 208. Optionally, the computing device can apply the therapeutic state to a 3D model of the portion of the subject's body (e.g. the subject's foot). For example, the 3D model can be manipulated or rotated along one or more axes that are specific to the muscular-skeletal deformity. The 3D model can be generated from the image(s) captured by the image capturing device and/or obtained from the data collected by the 3D scanner described in step 202. Then at 212, a therapeutic device (e.g., a brace or cast) can be designed. The therapeutic device can be designed based on the 3D model of the portion of the subject's body (e.g., the subject's foot) as manipulated to achieve the therapeutic state. The processing can optionally include the 3DMD Clubfoot Model Manipulation Process (CMMP) described below. For example, the 3D model can be trimmed, extruded, and patterned to create a 3D printing pattern. The therapeutic device (e.g., a brace or cast) can be printed using a 3D printer (e.g., the 3D printer 106 of FIG. 1) based on the 3D printing pattern. It should be understood that the therapeutic device designed as described above is customized for the subject. Optionally, the 3D model can be manipulated or rotated in accordance with a parameter unrelated to the deformity. The therapeutic device can be adjusted and/or redesigned after such manipulation. For example, the parameter unrelated to the deformity can account for growth of the subject and/or to otherwise adjust the therapeutic device for comfort of the subject.

As shown in FIG. 2, clubfoot can be treated with a series of adjustments—Correction 1 through Correction 1+n. The subject's deformed foot can be treated in a serial manner with a series of therapeutic devices (e.g., casts or braces) that incrementally adjust the subject's foot (e.g., by stretching muscles, ligaments, etc.) to correct the clubfoot deformity. For example, steps 202-212 in FIG. 2 can be repeated periodically such as weekly, bi-weekly, or monthly, for example. In other words, an image of the subject's foot is captured and processed to determine a quantitative measure of the clubfoot deformity. Using the quantitative measure, an incremental therapeutic state is determined for treating the clubfoot deformity, for example, by designing and applying a customized therapeutic device to achieve the incremental therapeutic state. After a period of treatment, another image of the subject's foot is captured, and the process is repeated to achieve the next incremental therapeutic state. This disclosure contemplates that the period can be any length of time and that the length between periods can have equal or unequal amounts of time.

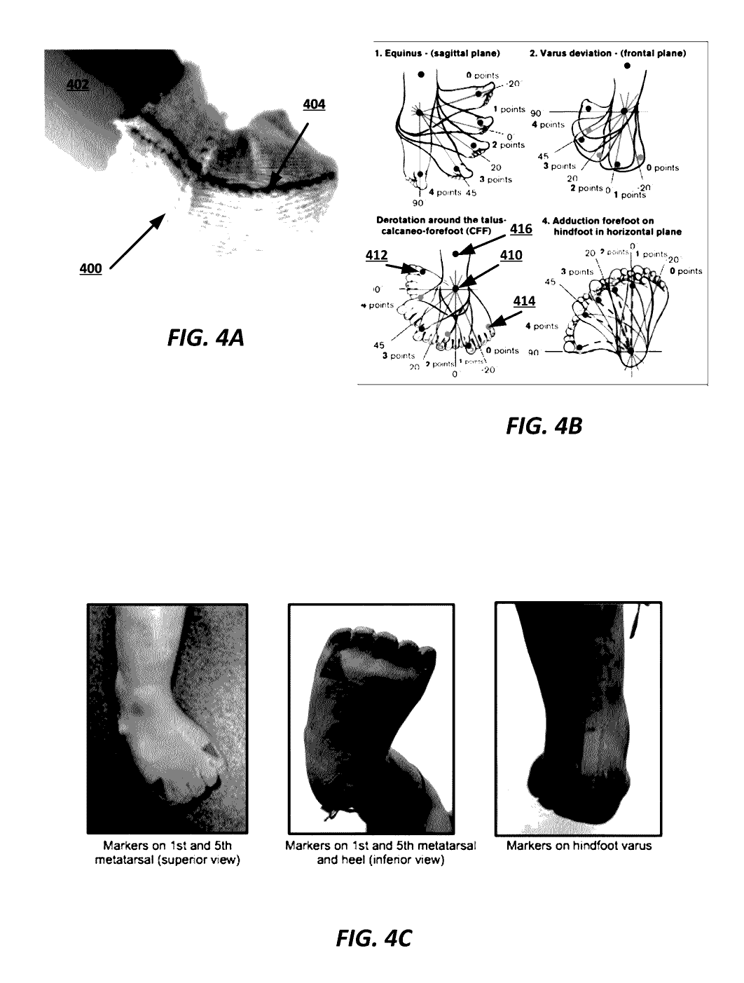

Optionally, the system can include a scoring sleeve with one or more markers arranged to aid in establishing the quantitative measure of the muscular-skeletal deformity (e.g., clubfoot). The scoring sleeve can be designed to be worn on the portion of the subject's body. Referring now to FIG. 4A, an example scoring sleeve 400 is shown. The scoring sleeve 400 is worn on a subject's foot 402, and the scoring sleeve 400 has a marker 404 arranged thereon. Optionally, in some implementations, the scoring sleeve can be a sock and/or other material designed to fit to the portion of the subject's body having the muscular-skeletal deformity. One objective of imaging is to locate the tripod of the foot, i.e., metatarsals I and V in the forefoot and calcaneus (heel) in the hind-foot. FIG. 4B is a diagram illustrating an example of locations where markers can be arranged on the scoring sleeve used to treat clubfoot, with the heel, first metatarsal head, and fifth metatarsal head marked from various angles in marker 410, marker 412, and marker 414, respectively. Accordingly, the markers can be arranged on the scoring sleeve to facilitate location of the tripod of the foot in the images. In addition, a marker 416 can optionally be placed along the upper leg to provide reference points for angle measurement when assessing parameters 1-3 in FIG. 4B. A line on the bottom of the foot will be used to assess adduction (parameter 4 in FIG. 4B), because no readily imagable reference points are visible from this angle. These deformity measurements can be combined with investigation of other potentially relevant deformity parameters (such as a direct cavus measurement), and dimensional measurements, also extracted from smartphone images. Referring now to FIG. 4C, another diagram illustrating an example of locations where markers can be arranged on the scoring sleeve used to treat clubfoot according to implementations described herein is shown. FIGS. 13A-13D illustrate a scoring sleeve imaged for each component of the deformity. FIG. 13A shows the cavus deformity. FIG. 13B shows the adductus deformity. FIG. 13C shows the varus deformity. FIG. 13D shows the equinus deformity.

It should be appreciated that the logical operations described herein with respect to the various figures may be implemented (1) as a sequence of computer implemented acts or program modules (i.e., software) running on a computing device (e.g., the computing device described in FIG. 5), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 5, an example computing device 500 upon which embodiments of the invention may be implemented is illustrated. It should be understood that the example computing device 500 is only one example of a suitable computing environment upon which embodiments of the invention may be implemented. It should be understood that the computing device 102, remote computing device 104, and/or the 3D printer 106 of FIG. 1 can include one or more components of the computing device 500 of FIG. 5. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 5 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

Described below is an example system and method for treatment of clubfoot. The system delivers 3D-printed corrective serial castings, custom fit to the subject's anatomy and therapeutic need using a parametric smartphone imaging modality. The treatment technique includes several aspects. First, the systems and methods include a smartphone-based imaging platform (e.g., remote computing device 104 of FIG. 1) for the registration of clubfeet. The user is guided through an imaging protocol for capturing the extent of each component of the clubfoot deformity with the smartphone camera. As described above, the captured image can optionally be a still image and/or a video (e.g., moving images). A scoring sleeve (e.g., scoring sleeve 400 of FIG. 4A) is worn on the subject's foot while the images are captured. The scoring sleeve includes one or more markers, which can be isolated and used by a 3DMD Parametric Clubfoot Assessment Process (PCAP) (e.g., an application running on computing device 102 of FIG. 1) to quantify the extent of component deformities. Second, the systems and methods transform clubfoot evaluation, typically a subjective process conducted by an experienced orthopedist, into an automated, quantitative procedure using 3DMD PCAP. The 3DMD PCAP automatically receives clubfoot images taken with a smartphone and classifies the extent of each constituent deformity parameter. The 3DMD PCAP is a quantitative approach to characterize deformity precisely and with high therapeutic relevance. Third, the systems and methods introduce a predictive casting technique (e.g., 3DMD Clubfoot Correction Predictive Process (CCPP) and Clubfoot Model Mapping Process (CMMP) (e.g., an application running on computing device 102 of FIG. 1)), which maps the parameters of current deformity to a 3D-printable cast model (e.g., printed using 3D printer 106 of FIG. 1) of the subsequent therapeutic state. By eliminating the current need to travel to specialized care providers for each casting, the systems and methods improve clubfoot treatment accessibility and enables more rapid deformity correction.

3DMD Parametric Clubfoot Analysis Process (PCAP)

Using the techniques described herein, it is possible to quantitatively assess or measure clubfoot deformity such that treatment can be automated. This disclosure contemplates that the quantitative measure can characterize a single component of the clubfoot deformity (e.g., the cavus, adduction, varus, or equinus component) alone. Alternatively, this disclosure contemplates that the quantitative measure can characterize more than one component of the clubfoot deformity. Alternatively or additionally, the respective quantitative measure for one or more of the components of the clubfoot deformity can be weighted, and the weighted quantitative measures can be summed to obtain an aggregate quantitative measure. As described herein, the quantitative measure for each of the components of the clubfoot deformity can be obtained by analyzing one or more images. The automated process can reduce or eliminate bias and/or other inter-evaluator error of conventional clubfoot treatment. 3DMD PCAP can be used to analyze one or more images of the portion of the subject's body having the muscular-skeletal deformity (e.g., the subject's foot). For example, the images can be taken by a caregiver of the subject having clubfoot, for example, using a smartphone or tablet computer. The subject can optionally be wearing a scoring sleeve having one or more markers arranged thereon. The analyzed images can then be relayed to a predictive treatment model (e.g., 3DMD Clubfoot Correction Predictive Process (CCPP)). An example 3DMD PCAP was implemented using MATLAB of MATHWORKS, INC. of NATICK, Mass.

The 3DMD PCAP can include receiving an image of the subject's deformed foot when the subject is wearing the scoring sleeve. As described above, the scoring sleeve has one or more markers that aid in the quantification of clubfoot parameters. It should be understood that a plurality of images of the subject's deformed foot can be received. Optionally, each of the images can capture one or more a plurality of components of the clubfoot deformity (e.g., the cavus, adduction, varus, and/or equinus components). Alternatively or additionally, the images can be a video.

Optionally, in some implementations, one or more images of a clubfoot deformity can be uploaded to a cloud storage folder synced locally to a computing device (e.g., the computing device 102 of FIG. 1) running the 3DMD PCAP. For example, a set of images can be uploaded to the cloud storage folder in JPEG format and bare filenames indicating each specific deformity, e.g. “cavus.jpg.” Optionally, each file set contains four images, each capturing a unique constituent deformity of the clubfoot and its corresponding markers on the scoring sleeve: varus, cavus, adductus, and equinus. This is shown in FIG. 3.

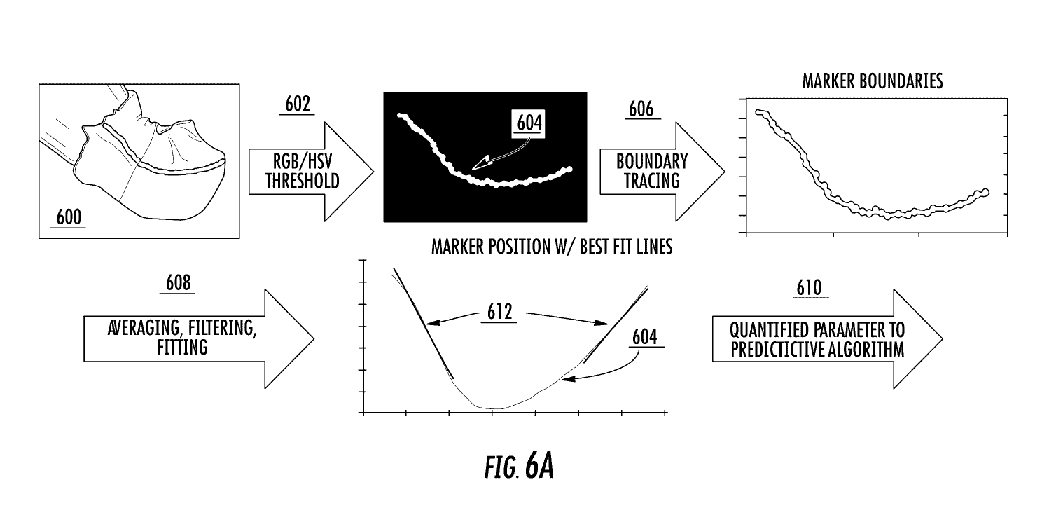

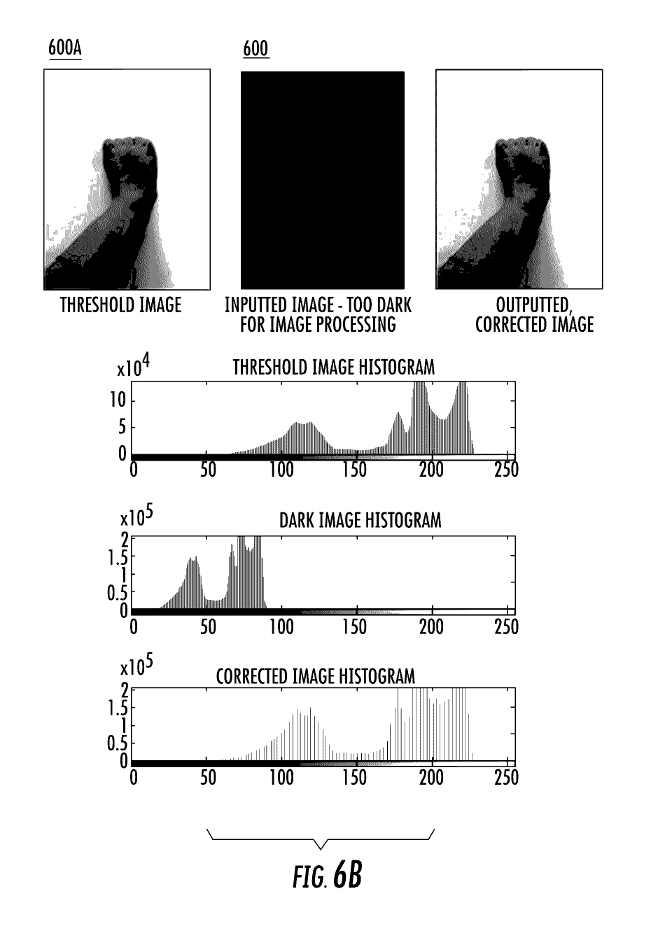

Referring now to FIGS. 6A-6B, for each image 600, the following steps can be performed to process and quantify the image and deformity:

Histogram Equalization: Each image 600 is standardized to a respective standard image 600A. The standard image 600A can optionally be stored locally on the computing device running the 3DMD PCAP. The hue, saturation, and value (HSV) or red, green, and blue (RGB) histogram of each image is equalized to the histogram of its respective standard.

Marker Isolation: At 602, one or more markers 604 of the scoring sleeve are isolated from images using RGB or HSV thresholding, or a combination of both. Threshold limits for each deformity can be calibrated using the standard image. Thresholding yields a binary patient image containing the isolated marker as well as artifacts remaining from thresholding. Combinations of erosions and dilations are used to decimate or break apart artifacts within the binary patient image, and close any gaps within the isolated marker. All remaining objects within the binary patient image are labeled and removed, save for the largest object in the binary image—the marker itself. Marker Tracing and Refining: At 606, the boundaries of the isolated marker are traced from the binary patient image. Marker boundaries are condensed into a single line tracing the extent of the deformity. Marker boundaries are averaged to a single line along the x axis. At 608, the line is detrended and a low pass filter is applied to remove inconsistencies.

Angle Calculation: At 610, the angular measure can be calculated. The angular measure can optionally be the quantitative measure of the clubfoot deformity. For example, least squares regression is used to linearly fit each end (e.g., 612) of the smoothed marker trace (e.g., 604) and the angle is computed. In particular, lines 612 that approximate ends of the marker 604 of the scoring sleeve within the image of the portion of the subject's body are traced, and an angular measure of the deformity is calculated based on the traced lines 612 (e.g., the angle between the two end lines).

Optionally, in some implementations, a cascade object detector can be used to automatically detect the clubfoot within an image and crop the image to that region only, eliminating as much background from the image as possible. The object detector can be trained with a series of “positive” images of clubfeet in the correct position and “negative” images objects the detector should not recognize. The object detector can be trained for each angle of the foot that must be imaged. By cropping the image to a limited region including the foot, it is possible to ensure that histogram equalization is not biased by unequal ratios of foot to background area.

3DMD Clubfoot Correction Predictive Process (CCPP)

Using the techniques described herein, it is possible to reduce or eliminate bias in clubfoot treatment and streamline the prescription of clubfoot correction. By determining treatment autonomously, the techniques described herein can reduce the need for the presence of a medical professional such as an orthopedic physician. This means less time required for the physician to attend to clubfoot patients, optimizing the time spent in the clinic.

As described above, the 3DMD PCAP can determine a quantitative measure of the muscular-skeletal deformity (e.g., clubfoot). The 3DMD CCPP can intake the quantitative measure determined by the 3DMD CPAP and predict or recommend a suggested course of treatment, e.g., the steps to follow for treatment of the clubfoot deformity. The 3DMD CCPP can therefore output the next incremental therapeutic state for treating clubfoot, which can serve as an input for the corrective manipulation process (e.g., the 3DMD Clubfoot Model Manipulation Process (CMMP)). An example 3DMD CCPP was implemented using MATLAB of MATHWORKS, INC. of NATICK, Mass.

As described above, the 3DMD CCPP uses the quantitative measure of the clubfoot deformity, which was obtained using the 3DMD CPAP described above. From this information regarding the current stage of treatment, the 3DMD CCPP can derive a set of instructions specifying the next stage (or next series of stages) of recommended manipulations. The next stage of treatment is also referred to herein as a “therapeutic state.” The therapeutic state can include the next recommended adjustment of the subject's foot for treating the clubfoot deformity. Instructions for the adjustment of the subject's foot can include a rate or amount of adjustment and/or a type of adjustment. The type of adjustment can be an adjustment directed to one or more of the components of the deformity (e.g., the cavus, adduction, varus, and/or equinus components of clubfoot deformity), including the order of the adjustments. Optionally, the set of instructions for clubfoot correction output by the 3DMD CCPP can be informed by one or more of the Ponseti method for clubfoot correction, the Dimeglio clubfoot scoring system, the Pirani method, and/or other information concerning treatment of clubfoot known in the art.

Optionally, the 3DMD CCPA can use the current clubfoot parameters (e.g., the quantitative measure determined by the 3DMD CPAP) and prescribe a next incremental correction based one or more of the following factors:

Treatment order: Clubfoot deformity components can be corrected in the following order for proper clubfoot treatment: cavus, adductus, varus, and equinus. Conventionally, treatment starts with correcting the pronation (rotation of the forefoot towards the floor relative to the midfoot). In other words, the forefoot is not twisted as far as the midfoot. Before the foot is rotated to its normal state, the forefoot and midfoot must be aligned so that the foot can be moved as a whole. Once this joint is lined up correctly, the next step is to straighten the cavus (high arch) and adductus (inward rotation of the forefoot). Lastly, the foot is rotated about the talus little by little, until the deformity is corrected. Once corrected, the foot must be braced in this position for one to two years so that the subtalar joint can form.

Amount of correction: The amount of correction for each incremental casting can be based on the quantitative measure, clinical data and/or other information known in the art.

Rate of treatment: The speed of correction with each incremental casting (e.g., the time spent in the cast) can be based upon the quantitative measure, clinical data and/or other information known in the art.

Physician feedback: A treating physician such as an orthopedic physician can provide input based on examination of the subject and/or images of the subject's foot.

Optionally, each component of the clubfoot deformity is associated with deformity parameter, including a “corrected” value and a rate of change. The 3DMD CCPP can return values of each parameter based on the quantitative measure output by 3DMD PCAP, which was based on the one or more images of the subject's foot. The 3DMD CCPP can run through the values one at a time and check whether each value meets the corrected threshold for the specific deformity parameter. If the parameter is corrected, the 3DMD CCPP can check the next parameter in treatment order—cavus, adductus, varus, equinus. If the parameter is not corrected, the 3DMD CCPP can apply a correction at the specified rate of change and supply instructions to apply that correction to the 3D model.

3DMD Clubfoot Model Manipulation Process (CMMP)

The 3DMD CMMP can accept a 3D model image (also referred to herein as a 3D model) of the clubfoot deformity and manipulate the 3D model image to incrementally correct the deformity. For example, the 3DMD CCPP can translate the numerical value of the severity of the clubfoot deformity (e.g., the quantitative measure) into a corrective state (e.g., the next, incremental therapeutic state), and the 3DMD CMMP can manipulate a 3D model to achieve the therapeutic state. A 3D model image can be generated from one or more images of the portion of the subject's body having the muscular-skeletal deformity (e.g., the subject's foot). Techniques for generating a 3D model from image(s) are known in the art and are therefore not described further herein. Alternatively, as described above, the subject's foot can be scanned using a 3D scanner, which generates the 3D modeling data. As described above, the 3D model can be manipulated to achieve the therapeutic state recommended by the 3DMD CCPP and/or other parameter unrelated to the deformity. Additionally, the 3DMD CMMP can use the manipulated 3D model to generate instructions (e.g., a 3D printing file) for printing a therapeutic device such as a brace or cast. An example 3DMD CMMP was implemented using AUTODESK MAYA of AUTODESK, INC. of SAN RAFAEL, Calif. (e.g., to manipulate the 3D model) and AUTODESK MESHMIXER of AUTODESK, INC. of SAN RAFAEL, Calif. (e.g., the design the therapeutic device).

After loading the 3D model of the subject's foot, the following steps can be performed to manipulate the 3D model:

Establish Bend Point: An axis (or axes) along which to manipulate the 3D model of the subject's foot can be selected. This axis or bend point corresponds with the deformity being corrected. For example, to correct the cavus component of clubfoot, the axis of curvature can be set at the apex of the foot arch.

Bend Foot: A predetermined angle can serve as the numerical input for the function. This angle is the degree to which the 3D model of the subject's foot is bent along the bend point and is directly inputted into the function. The result is a digital foot model with specific modifications that lead to the next stage of correction. The manipulated 3D model of the subject's foot can then be surface patterned.

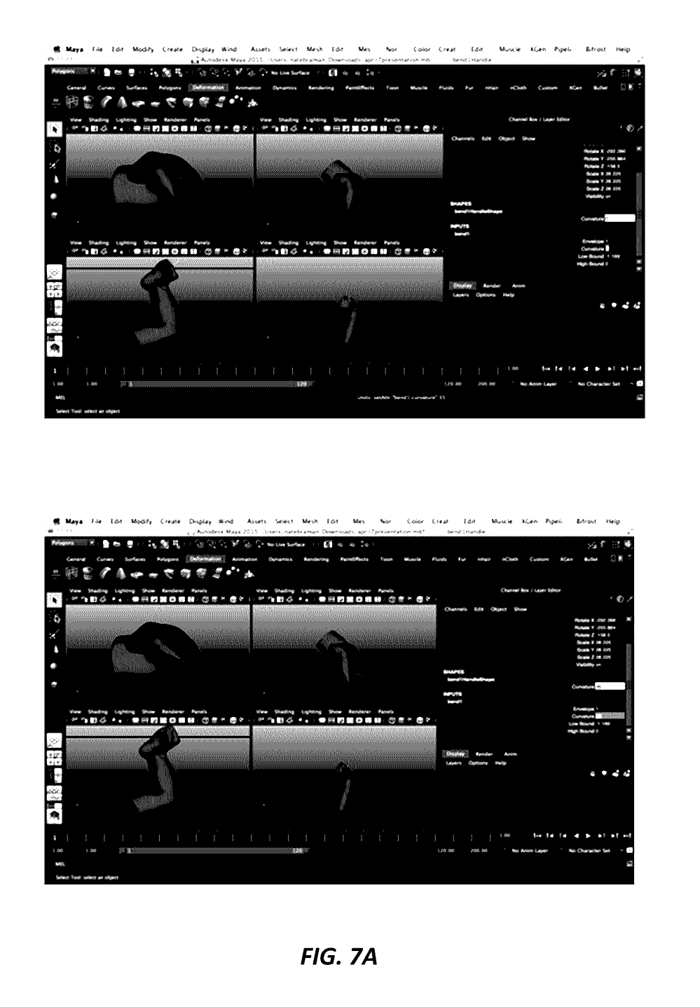

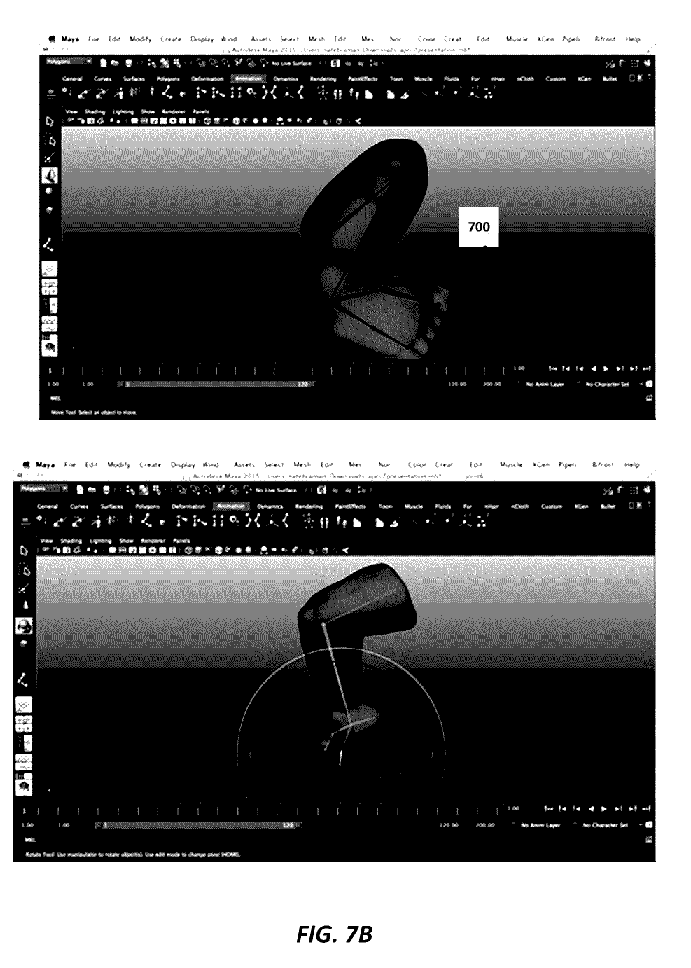

Referring now to FIG. 7A, screen captures illustrating manipulation of a 3D model of the subject's foot around an axis specific to the clubfoot deformity are shown. The “bend” feature was used to manipulate the 3D model of the foot in its current state along a specific axis to generate a next stage corrected foot. For example, in cavus correction, the axis of curvature was set at the apex of the foot arch. Once this axis was established, the “bend” of the foot is set to the numerical value for clubfoot correction as determined by the 3DMD CCPP as described above. The result is a digital 3D model with specific modifications that lead to the next stage of correction. As described below, a corresponding clubfoot cast for the next stage of correction is then generated based on the manipulated 3D foot model.

Referring now to FIG. 7B, screen captures illustrating manipulation of a 3D model of the subject's foot around the subject's joints are shown. Optionally, in some implementations, a skeleton can be placed within the 3D model of the subject's foot, which allows for manipulation of the 3D model in a manner closely matching conventional clubfoot treatment methods (e.g., Ponseti method). As shown in FIG. 7B, a skeleton 700 is drawn over the 3D model and then matched to the 3D model from multiple views. The 3D model and skeleton are bound together, allowing the 3D model to be manipulated along the joints of the skeleton.

As described above, a therapeutic device (e.g., cast or brace) can be designed based on the manipulated 3D model. For example, the therapeutic device can optionally be created from a patterned mesh file (e.g., also referred to herein as a “surface pattern”) of the 3D model as described below. The patterned mesh file or surface pattern can be created by hollowing and trimming the 3D model, extruding the outer layer (e.g., the shell) of the 3D model, and patterning the shell of the 3D model. The patterned mesh file can be converted into a 3D printing file, which can be used by a 3D printer to produce the therapeutic device.

Voronoi Filter and Point Generation: A system of points along the surface of the mesh can be created. The Voronoi filter is then applied over this set of points to create the pattern.

Filter Adjustment: The vertex quality selection function removes points from the mesh that fall outside of the numerical quality range chosen by the user. This is done using the inverted selection condition and will serve as the final patterned mesh.

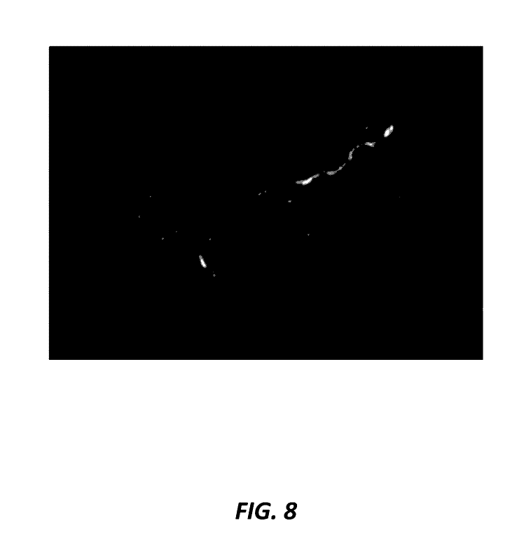

Pattern Isolation and Smoothing: All other layers are deleted aside from the inverted mesh, leaving a net-like surface. The inner circles of the mesh are smoothed using a Laplacian filter to get rid of jagged edges. The mesh is then used to create a cast, and example of which is shown in FIG. 8. This involves removing portions of the foot within the 3D model to get the shape of a short-leg cast. The ends of the cast are smoothed and closed to remove and sharp edges. The entire cast is then extruded to a given thickness before being split in half to create a clamshell. These manipulations still maintain the 3D model's inner dimensions so that the foot remains customized to the anatomical dimensions of the subject to whom the cast will be applied.

The 3DMD CMMP reduces the need for a physician being physically present to correct the subject's foot. For example, a physician could correct the deformity with the 3DMD CMMP and a cast could be created remotely and printed at any 3D printing location. This provides convenience to the subject in not having to see the physician frequently (e.g., weekly) since the course of treatment can be recommended (and optionally the cast created) automatically based on one or more images of the subject's foot.

### Other Examples

In some implementations, as described above, a 3D model image of the subject's foot is generated from the image(s) of the subject's foot each time images are captured. Alternatively, in some implementations and instead of reconstructing a 3D model image from scratch with each collection of images, it is possible to implement a parameterization method. This method would require fewer images, as only certain anatomical measurements (e.g., the quantitative measure described above) are needed to be extracted from images subsequent to creating the 3D model images. These values can then be used to manipulate the existing 3D model image (e.g., a 3D model generated from previously acquired images), personalizing it for the subject.

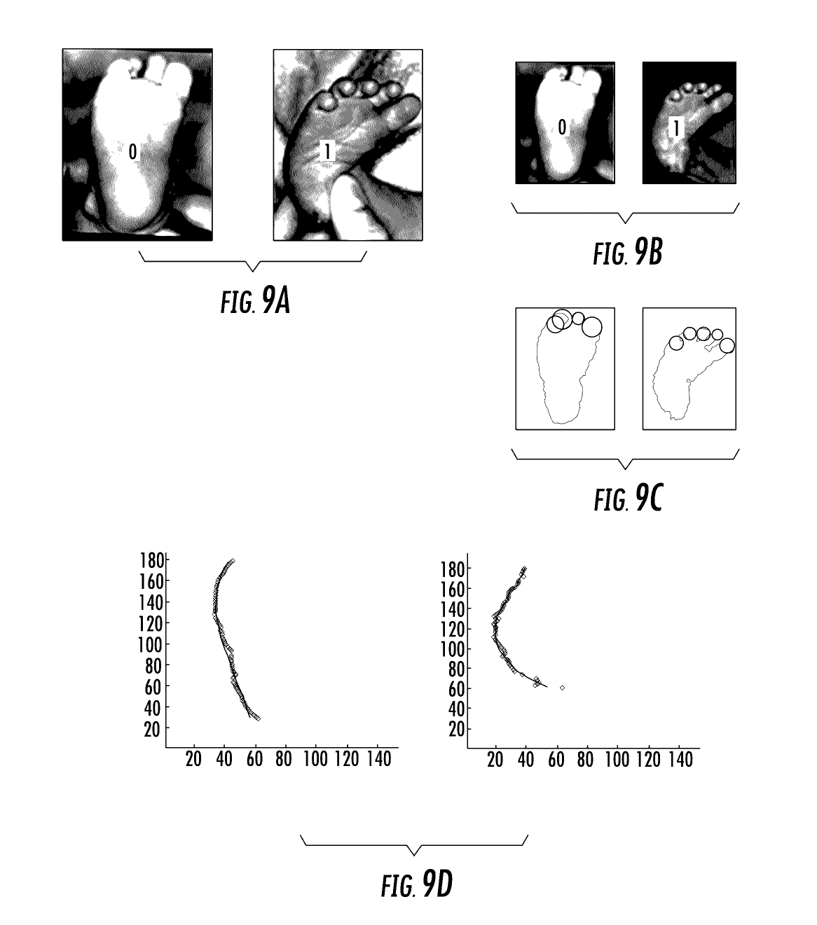

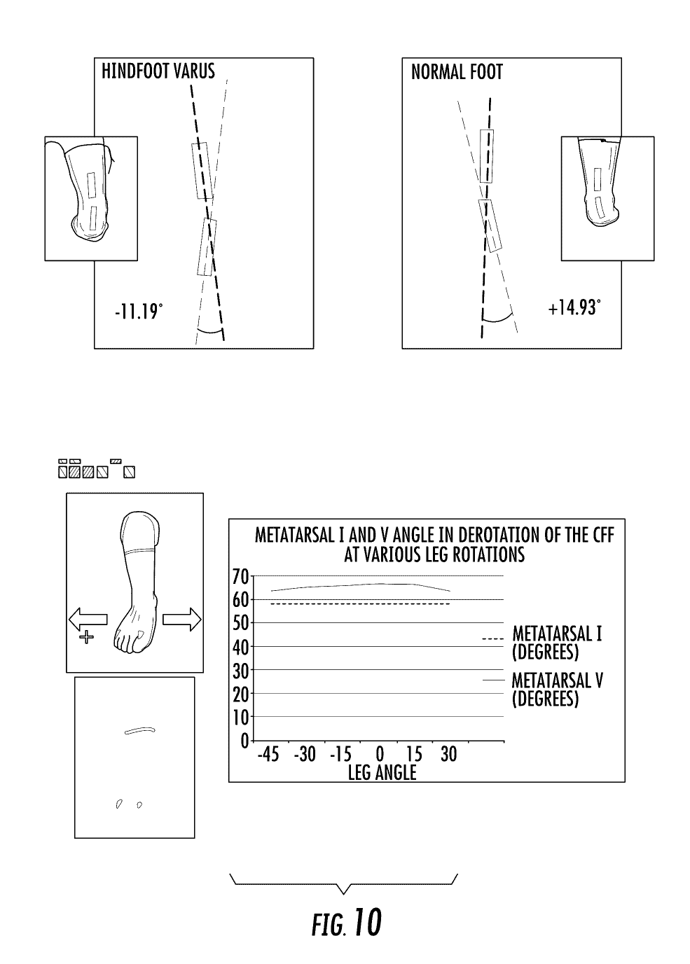

Referring now to FIGS. 9A-9D, example operations for calculating a quantitative measure of clubfoot deformity from images of the subject's foot are described. In particular, the operations describe calculating a quantitative measure of curvature of the lateral border (CLB), which is one of six Pirani parameters. Pirani parameters are a system for rating the severity of clubfoot deformities known in the art. For example, Pirani scoring is described by Dr. Muteti of Bethany Crippled Children's Hospital, Kijabe, in Clinical Management, Idiopathic Clubfoot, available at http://www.slideshare.net/alimenzah/ctev. This disclosure contemplates that other parameters, including but not limited to Pirani parameters, can be quantified using techniques described herein (e.g., see FIG. 10). FIG. 9A illustrates a Pirani scoring guide showing images of the least and most severe scoring CLB deformities (a “0” and “1” on the Pirani scale, respectively). FIG. 9B illustrates the Pirani scoring guide with increased contrast between foot and background and removal of a finger in one of the images. In FIGS. 9A and 9B, the deformity is scored without use of scoring sleeve. Circle detection was used to locate the toes, for later use during quantification. Images were also converted to binary and feet were segmented to a binary mask of the foot. FIG. 9C illustrates the Pirani scoring guide of FIG. 9A after circle detection and segmentation of the foot. The leftmost pixel location of binary images, from below the toes to bottom of the heel, were extracted and a third order order polynomial was fit using linear least squares to estimate CLB. FIG. 9D are graphs illustrating estimated CLB of the images in the Pirani scoring guide of FIG. 9A.

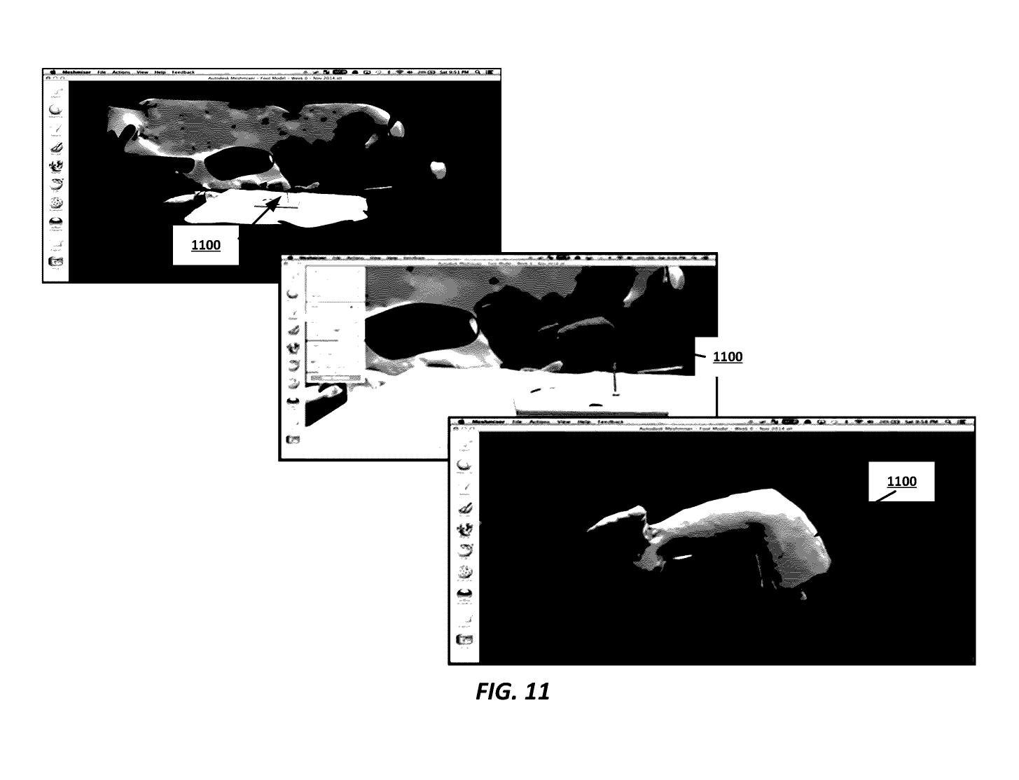

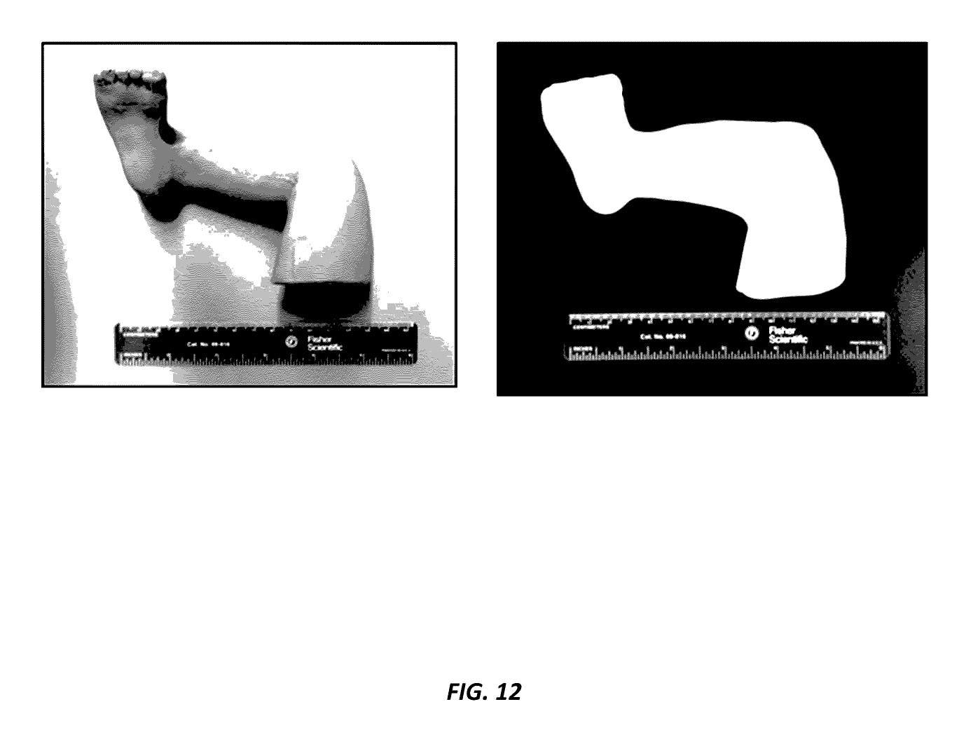

Referring now to FIG. 11, a 3D reconstruction 1100 (e.g., a 3D model) of a phantom foot having clubfoot deformity is described. The reconstruction was performed using AUTODESK 123D CATCH of AUTODESK, INC. of SAN RAFAEL, Calif. As shown in the upper-most screen capture of FIG. 11, a rough CAD rendering was produced, which included the imaging surroundings and the 3D model 1100. The 3D model 1100 was selected, isolated, and smoothed as shown in the middle and bottom-most screen captures of FIG. 11. Optionally, this disclosure contemplates restoring original dimensionality to the 3D model during reconstruction since the 3D model may not be properly scaled because modeling software does not translate physical dimensions to image dimensions. To spatially calibrate images, an adhesive indicator of known length can be placed on the subject's body to scale reconstructed models according to anatomical proportions. For example, to spatially calibrate 3D models, foot phantoms can be imaged with an adhesive scale marker, producing a 3D reconstruction of the phantom and marker. The marker can be traced and used to determine the relationship between digital and physical length, which can then be used to scale the model to proper dimensions. FIG. 12 illustrates a phantom foot having a clubfoot deformity (left image) and a 3D model of the phantom foot (right image) scaled spatially as described above. Optionally, a dimensional scaling algorithm can recognize the adhesive marker by a distinctive feature such as its color, measure the pixel length of the marker, and resize the 3D model accordingly.

The systems and methods described above provide clubfoot treatment using a custom 3D printed casting series that offers considerable improvements over conventional treatment:

Increased accessibility: patients no longer need to travel to a trained orthopedic surgeon for every casting.

Accelerated casting: Research has shown that clubfoot can be treated more quickly than current treatment periods, however frequency of casting is limited by the inconvenience of trips to the orthopod. The improved accessibility of assessment and rapid prototyping approach of the systems and methods described above allow casting patients up to 3× as frequently.

Parental involvement: The system and methods described above allows parents to take a greater role in their child's treatment.

Greater Cast Comfort/Convenience: 3D Printed casts allow for greater comfort than conventional plaster or soft casts, through improved breathability and visibility, customized fit, and reduced mass/size. These factors are especially important for infant patients, who must be carried and breast fed and lack the communication skills to vocalize that a fit may be overly tight and painful.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for establishing a serial treatment plan to correct a muscular-skeletal deformity of a subject, comprising:
receiving, using a computing device, an image of a portion of the subject's body having the muscular-skeletal deformity;
processing, using the computing device, the image of the portion of the subject's body to establish a quantitative measure of the muscular-skeletal deformity; and
determining, using the computing device, a therapeutic state to correct the muscular-skeletal deformity, wherein the therapeutic state includes an adjustment of the portion of the subject's body, and wherein at least one characteristic of the adjustment of the portion of the subject's body is related to the quantitative measure of the muscular-skeletal deformity.

2. The method of claim 1, wherein the at least one characteristic comprises an amount, a rate, or a type of adjustment of the portion of the subject's body.

3. The method of claim 1, further comprising determining, using the computing device, a plurality of therapeutic states to correct the muscular-skeletal deformity, wherein each therapeutic state includes a respective incremental adjustment of the portion of the subject's body.

4. The method of claim 1, further comprising applying a therapeutic device to the portion of the subject's body to achieve the therapeutic state.

5. The method of claim 1, further comprising:
generating, using the computing device, a 3D model image of the portion of the subject's body from the image of the portion of the subject's body;
manipulating, using the computing device, the 3D model image of the portion of the subject's body in accordance with the therapeutic state; and
designing, using the computing device, a therapeutic device for application to the portion of the subject's body to achieve the therapeutic state based on the manipulated 3D model image.

6. (canceled)

7. The method of claim 5, further comprising:
manipulating, using the computing device, the 3D model image of the portion of the subject's body in accordance with a parameter unrelated to the muscular-skeletal deformity; and
designing, using the computing device, an adjusted therapeutic device for application to the portion of the subject's body based on the manipulated 3D model image.

8. The method of claim 5, wherein the 3D model image of the portion of the subject's body is manipulated along an axis that is specific to the muscular-skeletal deformity, or wherein the 3D model image of the portion of the subject's body is simultaneously manipulated along a plurality of axes that are specific to the muscular-skeletal deformity.

9. (canceled)

10. The method of claim 5, further comprising generating, using the computing device, a surface pattern of the manipulated 3D model image of the portion of the subject's body, wherein the therapeutic device for application to the portion of the subject's body is designed based on the surface pattern.

11. The method of claim 10, further comprising:
generating, using the computing device, a 3D printing design model based on the surface pattern; and
transmitting, using the computing device, the 3D printing design model to a 3D printing device.

12. The method of claim 11, further comprising printing, using the 3D printing device, the therapeutic device based on the 3D printing design model.

13. (canceled)

14. The method of claim 1, wherein the quantitative measure comprises an angular measure of the muscular-skeletal deformity.

15. The method of claim 1, wherein the image of the portion of the subject's body includes a scoring sleeve that covers the portion of the subject's body, and wherein the scoring sleeve comprises a marker arranged to aid in establishing the quantitative measure of the muscular-skeletal deformity.

16. (canceled)

17. The method of claim 15, wherein processing, using the computing device, the image of the portion of the subject's body to establish a quantitative measure of the muscular-skeletal deformity further comprises:
tracing a line that approximates the marker of the scoring sleeve within the image of the portion of the subject's body, and
calculating an angular measure of the deformity based on the traced line.

18. The method of claim 17, wherein the traced line defines at least two ends, and the angular measure comprises an angle between the at least two ends of the traced line.

19-23. (canceled)

24. The method of claim 1, wherein the muscular-skeletal deformity is clubfoot.

25. A method for establishing a serial treatment plan to correct a muscular-skeletal deformity of a subject, comprising:
receiving, using a computing device, data for constructing a three-dimensional (3D) model of a portion of the subject's body having the muscular-skeletal deformity;
generating, using the computing device, a 3D model of the portion of the subject's body from the data;
establishing, using the computing device, a quantitative measure of the muscular-skeletal deformity based on the 3D model of the portion of the subject's body; and
determining, using the computing device, a therapeutic state to correct the muscular-skeletal deformity, wherein the therapeutic state includes an adjustment of the portion of the subject's body, and wherein at least one characteristic of the adjustment of the portion of the subject's body is related to the quantitative measure of the muscular-skeletal deformity.

26. (canceled)

27. A system for establishing a serial treatment plan to correct a muscular-skeletal deformity of a subject, comprising:
a computing device including a processor and a memory operably coupled to the processor, the memory having computer-executable instructions stored thereon that, when executed by the processor, cause the computing device to:
receive an image of a portion of the subject's body having the muscular-skeletal deformity;
process the image of the portion of the subject's body to establish a quantitative measure of the muscular-skeletal deformity; and
determine a therapeutic state to correct the muscular-skeletal deformity, wherein the therapeutic state includes an adjustment of the portion of the subject's body, and
wherein at least one characteristic of the adjustment of the portion of the subject's body is related to the quantitative measure of the muscular-skeletal deformity.

28. (canceled)

29. The system of claim 27, further comprising a remote computing device communicatively connected to the computing device over a network, wherein the remote computing device is configured to capture the image of the portion of the subject's body.

30. (canceled)

31. (canceled)

32. The system of claim 29, wherein the remote computing device is a smart phone or tablet.

33-41. (canceled)

42. The system of claim 27, further comprising a 3D printing device communicatively connected to the computing device over a network, wherein the 3D printing device is configured to print a therapeutic device for application to the portion of the subject's body.

43-77. (canceled)
